(19) United States
(12) Patent Application Publication
(10) Pub. No.: US 2005/0113394 A1
(43) Pub. Date: May 26, 2005

(54) Pharmaceutical compositions

(76) Inventors: Allen Wayne Wood, Durham, NC (US); Robin Currie, Durham, NC (US); Samir Chimanlal Mehta, Durham, NC (US); Gary Wayne Goodson, Durham, NC (US); Mickey Lee Wells, Durham, NC (US)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/997,400

(22) Filed: Nov. 24, 2004

Related U.S. Application Data
(60) Provisional application No. 60/525,630, filed on Nov. 26, 2003.

(51) Int. Cl. A61K 31/52; A61K 9/20

(52) U.S. Cl. 514/263.4; 424/464

(57) ABSTRACT

A pharmaceutical composition comprising (1S, cis)-4-[2-amino-6-(cyclopropylamino)-9H-purin-9-yl]-2-cyclopentene-1-methanol in an amount which achieves antiviral efficacy, a process for the preparation of such a composition, and a method of inhibiting human immunodeficiency virus (HIV) which comprises administering such a composition to an HIV infected patient is disclosed.

This application claims priority from U.S. Provisional Application 60/574,485 filed Nov. 26, 2003.

The present invention relates to pharmaceutical composition containing (1S, cis)-4-[2-amino-6-(cyclopropylamino)-9H-purin-9-yl]-2-cyclopentene-1-methanol useful in the treatment of diseases in mammals, including humans.

### BACKGROUND OF THE INVENTION

(1S, cis)-4-[2-amino-6-(cyclopropylamino)-9H-purin-9-yl]-2-cyclopentene-1-methanol (also known as abacavir, 1592U89, Ziagen®) and its antiviral use, particularly against HIV infections, is described in European Patent Specification Number 0434450. The succinate salt of (1S, cis)-4-[2-amino-6-(cyclopropylamino)-9H-purin-9-yl]-2-cyclopentene-1-methanol is described in WO96/06844. The hemisulfate salt of (1S, cis)-4-[2-amino-6-(cyclopropylamino)-9H-purin-9-yl]-2-cyclopentene-1-methanol is described in WO98/52949. Each of these references is incorporated herein by reference.

The success of modern multiple-drug treatments for HIV often requires strict compliance with a complex treatment regimen that can require the administration of many different drugs per day, administered at precisely timed intervals with careful attention to diet. Patient non-compliance is a well known problem accompanying such complex treatment regimens. Patient non-compliance is a critical problem in the treatment of HIV because such non-compliance may lead to the emergence of multiple-drug resistant strains of HIV.

The present invention addresses the issue of non-compliance by formulating an entire day's dose of (1S, cis)-4-[2-amino-6-(cyclopropylamino)-9H-purin-9-yl]-2-cyclopentene-1-methanol into a single solid dosage form, namely a tablet, to be given once daily. Simply combining two (2) current Ziagen 300 mg tablets into a single tablet would result in a tablet size too large to swallow without difficulty. Furthermore, the greater the amount of drug in the formulation the more excipients are needed in order to compress the mixture into an acceptable tablet. Increased amounts of some excipients can have adverse effects on tablet properties and can lead to problems of, for example, dissolution, content uniformity, hardness, and segregation.

At this high drug dose, it is difficult to compress a tablet to an acceptable size to administer to a patient. In order to achieve high drug loading in a tablet, the amount of traditional binders, diluents, and fillers that would be necessary to form the high drug dose into a tablet that exhibits content uniformity, appropriate hardness, appropriate dissolution characteristics, and that remains intact during manufacture and storage would lead to an unacceptable tablet size, namely a tablet with a total compression weight greater than 100 mg.

We have discovered that the addition of a highly compressible carrier to (1S, cis)-4-[2-amino-6-(cyclopropylamino)-9H-purin-9-yl]-2-cyclopentene-1-methanol allows the manufacture of tablets of acceptable size for administration to a patient. Furthermore, such tablets exhibit good content uniformity, hardness and dissolution characteristics.

### BRIEF DESCRIPTION OF THE INVENTION

The present invention provides pharmaceutical compositions comprising the active ingredient (1S, cis)-4-[2-amino-6-(cyclopropylamino)-9H-purin-9-yl]-2-cyclopentene-1-methanol, or pharmaceutically acceptable derivatives thereof, in the form of a tablet with high drug loading, while maintaining favorable tablet properties and suitable tablet size.

Another embodiment of the present invention is to provide a method for using these pharmaceutical compositions.

### DETAILED DESCRIPTION OF THE INVENTION

The present invention provides pharmaceutical compositions for one tablet delivering an entire day's dose of the active ingredient (1S, cis)-4-[2-amino-6-(cyclopropylamino)-9H-purin-9-yl]-2-cyclopentene-1-methanol or pharmaceutically acceptable derivatives thereof, in the form of a tablet with high drug loading, while maintaining favorable tablet properties and suitable tablet size.

Another embodiment of the present invention is to provide a method for using these pharmaceutical compositions.

The present invention includes a pharmaceutical composition, comprising:
a safe and therapeutically effective amount of (1S, cis)-4-[2-amino-6-(cyclopropylamino)-9H-purin-9-yl]-2-cyclopentene-1-methanol or a pharmaceutically acceptable derivative thereof; and
ii) a pharmaceutically acceptable highly compressible carrier.

The present invention also includes pharmaceutical compositions comprising a safe and therapeutically effective amount of (1S, cis)-4-[2-amino-6-(cyclopropylamino)-9H-purin-9-yl]-2-cyclopentene-1-methanol (herein referred to as “abacavir”) or a pharmaceutically acceptable derivative thereof and a pharmaceutically acceptable highly compressible carrier wherein the composition has a volume in the range of 0.7-1.0 mL. Further pharmaceutical compositions of the present invention comprise abacavir as described above wherein the composition exhibits acceptable tablet hardness, for example of greater than 20 kilopounds at 25 kilonewtons of force for a 1075 mg tablet.

The phrase “safe and therapeutically effective amount,” as used herein, means a sufficient amount of a drug, compound, composition, product or pharmaceutical agent to abate or reverse or treat a malady in a human or other mammal without severely harming the tissues of the mammal to which the drug or pharmaceutical agent is administered.

The phrase “pharmaceutically acceptable derivative,” as used herein, means any pharmaceutically acceptable salt, solvate, ester, or salt of such ester, or any other compound which, upon administration to the recipient, is capable of providing (directly or indirectly) the intended active ingredient or any active metabolite or residue thereof.

The phrase “pharmaceutically acceptable derivative of abacavir” as used herein, means any pharmaceutically acceptable salt, solvate, ester, or salt of such ester, of abacavir, or any other compound which, upon administration to the recipient, is capable of providing (directly or indirectly) abacavir or any antivirally active metabolite or residue thereof. A preferred pharmaceutically acceptable derivative of abacavir is abacavir hemisulfate salt.

The phrase “highly compressible carrier” as used herein means binder or filler that provides good tableting properties such as tablet hardness, low friability, and flow at quantities significantly lower than conventional fillers or binders such as Avicel® PH 101, Avicel® PH102, lactose, and other similar binders or fillers.

The phrase “drug loading,” as used herein, means the ratio of drug to total weight of tablet.

The pharmaceutical compositions of the present invention contain highly compressible carriers, for example, diluents, binders or fillers, for example, highly compressible microcrystalline cellulose. The advantages of highly compressible microcrystalline cellulose are low bulk density and high compressibility, superior compatibility and low friability. The use of highly compressible microcrystalline cellulose enables compaction at lower forces and results in the capability to manufacture harder tablets. The carrier(s) must be pharmaceutically acceptable in the sense of being compatible with the other ingredients of the formulation and not deleterious to the recipient thereof.

The compositions of the present invention employ safe and therapeutically effective amounts of abacavir or a pharmaceutically acceptable derivative thereof along with a safe and effective amount of a pharmaceutically acceptable highly compressible carrier. Highly compressible carriers may be diluents, binders, or fillers. Examples include, but are not limited to, highly compressible microcrystalline cellulose, for example, Ceolus®, ProSolv™, and Avicel®PH105 microcrystalline cellulose.

The present invention further includes a pharmaceutical composition consisting essentially of abacavir, or a pharmaceutically acceptable derivative thereof, and Ceolus® microcrystalline cellulose.

Compositions of the present invention include unit dosage forms, for example, tablets containing abacavir wherein the tablet has a volume of less than 1.3 mL, advantageously less than 1.0 mL, or in the range of 0.7-1.0 mL, preferably about 0.9 mL. Tablets of the present invention containing abacavir exhibit properties that are advantageous for administration as a pharmaceutical composition. For example, tablets of the present invention may have a thickness of less than or equal to 7.6 mm, may exhibit low friability (<0.3%), for example, a friability of less than or equal to 0.1%, may exhibit a hardness of greater than 18 kilopounds for a 1075 mg tablet, and/or may exhibit a disintegration of less than or equal to 10 minutes, advantageously less than or equal to 2 minutes.

The present invention includes pharmaceutical compositions as described above which are flowable, compressible, have low friability, good disintegration times, good tablet hardness, and acceptable dissolution.

The present invention also includes pharmaceutical compositions comprising abacavir or a pharmaceutically acceptable derivative thereof, and Ceolus® microcrystalline cellulose. Such compositions may have a volume of about 0.9 mL and/or exhibit a hardness of greater than 18 kilopounds and/or exhibit a disintegration of less than or equal to 1 minutes, advantageously less than or equal to 2 minutes.

The present invention includes a pharmaceutical composition comprising abacavir, or a pharmaceutically acceptable derivative thereof in an amount from about 20% to 80% of total compression weight or from about 30% to about 70% of total composition weight. The pharmaceutical composition may advantageously be in the form of a tablet, said tablet having 20%-80% drug loading or 30% to 60% drug loading, advantageously 40% to 60% drug loading.

Another embodiment of the present invention is to simplify treatment regimens for HIV and other viruses with the goal of enhancing patient compliance by providing a simplified dosage form containing pharmaceutically acceptable amounts of abacavir or pharmaceutically acceptable derivatives thereof.

The present invention also includes a method for treating, reversing, reducing or inhibiting retroviral infections in particular HIV infections, in a mammal, in particular a human, which method comprises administering to said mammal a safe and effective amount of a composition according to the invention.

The present invention provides the use of abacavir, or a pharmaceutically acceptable derivative thereof, and a pharmaceutically acceptable highly compressible carrier in the manufacture of a medicament for the treatment of a retroviral infection, in particular an HIV infection. Preferably, the medicament is provided as a once-daily dosing regimen.

It will be appreciated by those skilled in the art that reference herein to “treatment” extends to both the prophylaxis and the treatment of an established malady, infection or its symptoms.

The compositions of the present invention may optionally employ a safe and effective amount of a diluent, a safe and effective amount of a disintegrant, and a safe and effective amount of a lubricant or any other safe and effective amounts of excipients commonly used in the art.

The compositions of the present invention may include from 0 to about 2% magnesium stearate; from about 0.0 to about 5% glidant; from 0 to about 5% sodium starch glycolate; and from about 15 to about 50% microcrystalline cellulose.

The pharmaceutical compositions of the present invention may optionally contain silicon dioxide (SiO₂), also referred to as colloidal silica, fumed silicon dioxide, fumed silica, light anhydrous silicic acid, silicic anhydride, AEROSIL™ or CAB-O-SIL™; asbestos free talc, sodium aluminosilicate, calcium silicate, powdered cellulose, microcrystalline cellulose, corn starch, sodium benzoate, calcium carbonate, magnesium carbonate, metallic stearates, calcium stearate, magnesium stearate, zinc stearate, stearowet C, starch, starch 1500, magnesium lauryl sulfate, magnesium oxide, colloidal silicon dioxide in combination with microcrystalline cellulose or ProSolv™.

Abacavir may be prepared by the method described in European Patent Specification Number 0434450 or WO95/21161, which are incorporated herein by reference hereto. The succinate salt of 1592U89 may be prepared by the method described in WO96/06844, which is incorporated herein by reference hereto. The hemisulfate salt of 1592U89 may be prepared by the method described in WO98/52949, which is incorporated herein by reference hereto. Preferred salts of abacavir include the succinate salt and the hemisulfate salt.

The invention is preferably presented as a pharmaceutical composition suitable for oral administration. Such compositions may conveniently be presented as discrete units such as tablets, caplets, capsules, or any other form suitable for oral administration and compatible with the compositions of the present invention, each containing a predetermined amount of the active ingredient. A particularly suitable composition may be prepared from direct compression or granulation processes. Such compositions may contain safe and effective amounts of conventional excipients such as binding agents, fillers, lubricants, or disintegrants. The tablets may also be coated according to any method known to persons skilled in the art that would not interfere with the tablets' release properties, or the other physical or chemical characteristics of the present Invention. Tablet coating is further described and delineated by Remington, The Science & Practice of Pharmacy 19th ed. 1995 incorporated herein by reference. When desired, the above formulations may also be modified by any method known to persons skilled in the art to achieve sustained release of active ingredients. The compositions may also include a safe and effective amount of other active ingredients, such as antimicrobial agents or preservatives.

These compositions of the present invention are suitable for administration to humans or other mammals particularly via an oral route of administration. However, other routes as utilised by medical practitioners and others skilled in the art of pharmaceutical dosage administration such as pharmacists and nurses are not foreclosed.

It will be appreciated by those skilled in the art that the amount of active ingredients required for use in treatment will vary according to a variety of factors, including the nature of the condition being treated and the age and condition of the patient, and will ultimately be at the discretion of the attending physician, veterinarian or health care practitioner.

In general, however, a suitable dose of abacavir for administration to a human for treatment of an HIV injection may be in the range of 0.1 to 120 mg per kilogram body weight of the recipient per day, preferably in the range of 3 to 90 mg per kilogram body weight per day and most preferably in the range 5 to 60 mg per kilogram body weight per day.

Compositions of the present invention enable patients greater freedom of use from varied multiple dosage medication regimens and also ease the needed diligence required in remembering complex daily dosing times and schedules by allowing once daily dosing of abacavir.

The compositions of the present invention conveniently allow administration of one compound in unit dosage form containing, for example, from about 15 to about 1200 mg of abacavir, particularly from about 100 to about 750 mg of abacavir, and most particularly about 600-700 mg of abacavir. The composition of the present invention may be used in combination with other pharmaceutical formulations as a component of a multiple drug treatment regimen.

Compositions of the present invention may also be packaged as articles of manufacture comprising a safe and therapeutically effective amount of abacavir, or a pharmaceutically acceptable derivative thereof; and a safe and effective amount of a pharmaceutically acceptable highly compressible carrier.

Any of the various methods known by persons skilled in the art for packaging tablets, caplets, or other solid dosage forms suitable for oral administration, that will not degrade the components of the present invention, are suitable for use in packaging. Tablets, caplets, or other solid dosage forms suitable for oral administration, may be packaged and contained in various packaging materials particularly glass and plastic bottles and also including unit dose blister packaging. The packaging material may also have labelling and information related to the pharmaceutical composition printed thereon. Additionally, an article of manufacture may contain a brochure, report, notice, pamphlet, or leaflet containing product information. This form of pharmaceutical information is referred to in the pharmaceutical industry as a “package insert.” A package insert may be attached to or included with a pharmaceutical article of manufacture. The package insert and any article of manufacture labelling provides information relating to the pharmaceutical composition. The information and labelling provides various forms of information utilised by health-care professionals and patients, describing the composition, its dosage and various other parameters required by regulatory agencies such as the United States Food and Drug Agencies.

The compositions of the present invention can be formulated using methods and techniques suitable for the compositions physical and chemical characteristics and that are commonly employed by persons skilled in the art in preparing oral dosage forms utilising direct compression or granulation processes. Remington, The Science & Practice of Pharmacy, p. 1615-1623, 1625-1648, and other applicable sections, 19th ed. (1995).

Compositions of the present Invention in their method aspect are administered to a human or other mammal in a safe and effective amount as described herein. These safe and effective amounts will vary according to the type and size of mammal being treated and the desired results of the treatment.

### EXAMPLES

The following examples further describe and demonstrate particular embodiments within the scope of the present Invention. The examples are given solely for illustration and are not to be construed as limitations as many variations are possible without departing from spirit and scope of the Invention.

### Example 1

Tablet Containing Abacavir for Once-Daily (“OD”) Dosing

| | Quantity | Quantity |
|---|---|---|
| Component | (mg/tablet) | (% w/w) |
| Abacavir Hemisulfate | 702.0 | 65.3 |
| Ceolus ® | 323.55 | 30.1 |
| Sodium Starch Glycolate | 43.00 | 4.00 |
| Magnesium Stearate | 6.45 | 0.60 |
| Total Tablet Weight | 1075 | |

Bulk Preparation Method

The quantities of the present example of manufacturing procedure are based on a typical batch size of 300 kg and may be adjusted depending on batch size.

First the components are weighed from bulk containers in the following amounts:

| Ingredients | Amount (kg) |
|---|---|
| Abacavir hemisulfate | 195.9 |
| Ceolus ® (Microcrystalline Cellulose, NF) | 90.3 |
| Sodium Starch Glycolate | 12.0 |
| Magnesium Stearate | 1.8 |

The components are then sieved using a Russel-SIV equipped with a 14 mesh (1.4 mm opening) or an equivalent sieve and mesh, and deposited into a stainless-steel blending container.

The abacavir, Ceolus®, and sodium starch glycolate, NF are blended for 12 minutes using a suitable blender, such as a Matcon-Buls bin-type blender, a V-blender or equivalent. The magnesium stearate is then added to the mixture and blending is continued for approximately 1 to 2 minutes.

The lubricated blend is then compressed using a suitable rotary tablet press, typically a Fette 2090 or equivalent. In-process controls for tablet weight and hardness are applied at appropriate intervals throughout the compression run and adjustments to the tablet press are made as necessary.

### Example 2

Comparative Batch Data for Different Carriers/Binders

Tablets were weighed on an analytical balance. A digital caliper was used to measure the thickness of the tablets. Tablet hardness was measured on a suitable hardness tester by placing the tablets lengthwise between the crushing jaws. Powder flow was determined by placing a powder sample into a Flodex™. The sample was then allowed to sit undisturbed for fifteen seconds prior to being discharged through a stainless steel orifice. The orifices were changed as needed until the smallest size was determined that allowed the powder to flow freely. Friability and disintegration was measured according to the current U.S. Pharmacopeia (USP 25-NF 20).

| Test | Ceolus | Avicel PH101 |
|---|---|---|
| Compression Weight (mg) | 1075 | 1075 |
| Thickness (mm) | 7.04 | 7.16 |
| Friability (%) | 0.0 | 0.02 |
| Hardness (kp) | 28.0 | 16.5 |
| Disintegration (min) | 0.5 | 0.5 |
| Flow (mm) | 17.0 | 9.0 |

Acceptable attributes based on data above:

| | | Avicel ® | Avicel ® | |
|---|---|---|---|---|
| Test | Ceolus ® | PH101 | PH105* | Prosolv ™* |
| Friability | yes | yes | yes | yes |
| Hardness | yes | no | yes | yes |
| Disintegration | yes | yes | yes | yes |
| Flow | yes | yes | marginal | yes |
| Appearance | yes | yes | yes | yes |
| Tooling | yes | no | yes | yes |
| Apperance | | | | |

*Note responses are based on dry lab estimates

The application of which this description and claims form part may be used as a basis for priority in respect of any subsequent application. The claims of such subsequent application may be directed to any embodiment or combination of embodiments described herein. They may take the form of product, composition, process or use claims and may include, by way of example and without limitation, one or more of the following claims.

1. A pharmaceutical composition comprising:
i) a safe and therapeutically effective amount of (1S, cis)-4-[2-amino-6-(cyclopropylamino)-9H-purin-9-yl]-2-cyclopentene-1-methanol or a pharmaceutically acceptable derivative thereof; and
ii) a pharmaceutically acceptable highly compressible carrier.

2. A pharmaceutical composition comprising:
i) a safe and therapeutically effective amount of (1S, cis)-4-[2-amino-6-(cyclopropylamino)-9H-purin-9-yl]-2-cyclopentene-1-methanol; and
ii) a pharmaceutically acceptable highly compressible carrier
wherein said composition has a volume in the range of 1.0-1.3 mL.

3. A pharmaceutical composition in tablet form comprising:
i) a safe and therapeutically effective amount of (1S, cis)-4-[2-amino-6-(cyclopropylamino)-9H-purin-9-yl]-2-cyclopentene-1-methanol or a pharmaceutically acceptable derivative thereof; and
ii) a pharmaceutically acceptable highly compressible carrier
wherein said composition exhibits a tablet hardness of greater than 18 kilopounds at 25 kilonewtons of force.

4. A pharmaceutical composition according to claim 1, wherein the pharmaceutically acceptable highly compressible carrier is selected from a group consisting of diluents, binders, and fillers.

5. A pharmaceutical composition according to claim 4 wherein the pharmaceutically acceptable highly compressible binder is selected from the group consisting of highly compressible microcrystalline cellulose.

6. A pharmaceutical composition according to claim 5 wherein the compressible microcrystalline cellulose is Ceolus® microcrystalline cellulose.

7. A pharmaceutical composition according to claim 1 wherein said (1S, cis)-4-[2-amino-6-(cyclopropylamino)-9H-purin-9-yl]-2-cyclopentene-1-methanol, or pharmaceutically acceptable derivative thereof, is present in an amount of 20% to 80% of total composition weight.

8. A pharmaceutical composition according to claim 1 wherein the amount of (1S, cis)-4-[2-amino-6-(cyclopropylamino)-9H-purin-9-yl]-2-cyclopentene-1-methanol, or a pharmaceutically acceptable derivative thereof, is from about 15 to about 1200 mg per unit dosage form.

9. The pharmaceutical composition according to claim 1 wherein the pharmaceutically acceptable derivative of (1S, cis)-4-[2-amino-6-(cyclopropylamino)-9H-purin-9-yl]-2-cyclopentene-1-methanol is the hemisulfate salt.

10. The pharmaceutical composition according to claim 1 wherein the pharmaceutically acceptable highly compressible carrier is present in an amount of 5% to about 50% by weight.

11. A pharmaceutical composition comprising (1S, cis)-4-[2-amino-6-(cyclopropylamino)-9H-purin-9-yl]-2-cyclopentene-1-methanol, or a pharmaceutically acceptable derivative thereof and Ceolus® microcrystalline cellulose.

12. A pharmaceutical composition consisting essentially of (1S, cis)-4-[2-amino-6-(cyclopropylamino)-9H-purin-9-yl]-2-cyclopentene-1-methanol, or a pharmaceutically acceptable derivative thereof and Ceolus® microcrystalline cellulose.

13. A pharmaceutical composition according to claims 11 wherein the pharmaceutically acceptable derivative of (1S, cis)-4-[2-amino-6-(cyclopropylamino)-9H-purin-9-yl]-2-cyclopentene-1-methanol is the hemisulfate salt.

14. A pharmaceutical composition according to claim 3 wherein the composition has a volume in the range of 1.0-1.3 mL.

15. A pharmaceutical composition according to claim 1 in the form of a tablet.

16. A pharmaceutical composition according to claim 1 for once daily administration.

17. A pharmaceutical composition according to claim 1 wherein the composition is coated with a pharmaceutically acceptable coating.

18. A method for maintaining high drug loading of an abacavir pharmaceutical composition by including a safe and effective amount of a pharmaceutically acceptable highly compressible carrier.

19. A method according to claim 18 wherein the highly compressible carrier is highly compressible microcrystalline cellulose.

20. A method for treating, reversing, reducing or inhibiting retroviral infections by administering a safe and effective amount of a composition according to claims 1.

21. The method for treating, reversing, reducing or inhibiting retroviral infections according to claim 20, wherein the retrovirus is HIV.

22. (canceled)

23. An article of manufacture comprising:
i) packaging material; and
ii) a pharmaceutical composition contained within the packaging material, comprising:
a) a safe and therapeutically effective amount of (1S, cis)-4-[2-amino-6-(cyclopropylamino)-9H-purin-9-yl]-2-cyclopentene-1-methanol or a pharmaceutically acceptable derivative thereof; and
b) a pharmaceutically acceptable highly compressible carrier
wherein friability, hardness and disintegration of the resulting composition is maintained.

24. An article of manufacture according to claim 23 additionally comprising a brochure containing product information.

25. An article of manufacture according to claim 23 wherein the packaging material is unit dose blister packaging.

26. A process for the preparation of a pharmaceutical composition as claimed in claim 1 which process comprises admixture of (1S, cis)-4-[2-amino-6-(cyclopropylamino)-9H-purin-9-yl]-2-cyclopentene-1-methanol or a pharmaceutically acceptable derivative thereof, and a pharmaceutically acceptable highly compressible carrier.
